(19) United States
(12) Patent Application Publication
(10) Pub. No.: US 2021/0338878 A1
(43) Pub. Date: Nov. 4, 2021

(54) UV LED Face Mask Attachment

(71) Applicant: Martin Buskirk, Apalachicola, FL (US)

(72) Inventor: Martin Buskirk, Apalachicola, FL (US)

(21) Appl. No.: 17/244,669

(22) Filed: Apr. 29, 2021

Related U.S. Application Data
(60) Provisional application No. 63/018,907, filed on May 1, 2020.

(51) Int. Cl. A61L 9/20; A41D 13/11; A62B 7/10; A62B 23/02; A62B 18/08

(52) U.S. Cl. CPC A61L 9/20; A41D 13/11; A62B 7/10; A61L 2209/14; A62B 18/08; A61L 2209/12; A61L 2209/15; A62B 23/02

(57) ABSTRACT

A face mask attachment and a face mask made to disinfect air flowing in and out of a face mask with ultraviolet radiation. The face mask attachment has a disinfecting unit and a modular attachment system that removably attaches the disinfecting unit to any face mask. The face mask has a face covering portion, a disinfecting unit, and modular attachment system. Furthermore, the disinfecting unit includes a UVC LED, a circuit to operate the UVC LED, and a housing to encloses the circuit and the UVC LED.

This application claim priority to U.S. Provisional Application Ser. No. 63/018,907 filed on May 1, 2020 and entitled “Protective Facial Mask with UV-C Led,” which is incorporated herein, in its entirety, by reference.

### FIELD

The exemplary embodiments describe a face mask attachment, and more specifically a disinfecting face mask attachment that utilizes a clip or a modular attachment system to attach to a face mask in a removeable manner and ultraviolet (UV) radiation to disinfect the air coming in and out of the face mask.

### BACKGROUND INFORMATION

UV radiation has been employed as a disinfecting agent in various applications including protective face masks. UV radiation has been incorporated into protective face masks to disinfect the air coming in and out, which is typically done with a filter system that has a built-in UV radiation source, e.g., the overall UV filter system cannot be detached from the protective face mask. Thus, when the protective face mask becomes unwearable, the UV filter system becomes useless, e.g., the user cannot wash the mask.

### SUMMARY

Some exemplary embodiments are related to a face mask attachment having a disinfecting unit that includes an ultraviolet-C light emitting diode, a circuit configured to operate the ultraviolet-C light emitting diode, and a housing adapted to enclose the circuit and the ultraviolet-C light emitting diode. The face mask attachment further includes a modular attachment system configured to removably attach the disinfecting unit to a face mask.

Other exemplary embodiments are related to a face mask having a face covering portion, a disinfecting unit and a modular attachment system configured to releasably attach the disinfecting unit to the face covering portion. The disinfecting unit includes an ultraviolet-C light emitting diode, a circuit configured to operate the ultraviolet-C light emitting diode, and a housing adapted to enclose the circuit and the ultraviolet-C light emitting diode.

### BRIEF DESCRIPTION OF THE DRAWINGS

FIG. 1 shows a side view of a face mask with a disinfecting unit according to various exemplary embodiments.

FIG. 2 shows a front view of the disinfecting unit, wherein the housing compartment has a cylindrical shape according to various exemplary embodiments.

FIG. 3 shows a rear exploded view of a modular attachment system used to removably attach the disinfecting unit to a face mask according to various exemplary embodiments.

FIG. 4 shows a front exploded view of the disinfecting unit according to various exemplary embodiments.

FIG. 5 shows a side view of a disinfecting unit with a filter system attached to a face mask according to various exemplary embodiments.

FIG. 6 shows a front exploded view of a disinfecting unit adapted to include a filter system according to various exemplary embodiments.

FIG. 7 shows a rear exploded view of a disinfecting unit with a filter system, wherein the circuit is configured to sit under an inner cover according to various exemplary embodiments.

FIG. 8 is a rear view of a face mask that has a disinfecting unit attached to the inside of the mask, wherein the inner cover may be configured to hold the UV LED in place according to various exemplary embodiments.

FIG. 9 shows a front view of an outer cover of a disinfecting unit attached to the front of a protective face mask according to various exemplary embodiments.

FIG. 10 shows a diagram of a circuit that could be used to power a UV LED and an operating light according to various exemplary embodiments.

### DETAILED DESCRIPTION

The exemplary embodiments may be further understood with reference to the following description and the related appended drawings, wherein like elements are provided with the same reference numerals. The exemplary embodiments describe a face mask attachment that may include a disinfecting unit and a clip or a modular attachment system, wherein the disinfecting unit includes an ultraviolet light emitting diode (UV LED) enclosed in a housing compartment. The UV LED may be powered by, for example, a disposable battery, rechargeable battery, or power cord connected to a mobile phone or battery pack.

The exemplary embodiments may be configured to attach to a variety of face masks through a clip or modular attachment system. The exemplary embodiments may also be configured to include a filter system that includes a filter ring and a filter element. Additionally, exemplary embodiments may also include a power switch that controls the UV LED and an operating light to notify the user when the UV LED is operating. Each of these exemplary embodiments will be described in greater detail below.

Throughout this description, exemplary dimensions will be provided for various components of the exemplary embodiments. It should be understood that these dimensions are only exemplary and are provided to give a general size for the various components. The exemplary embodiments are not limited to components having the exemplary dimensions provided herein.

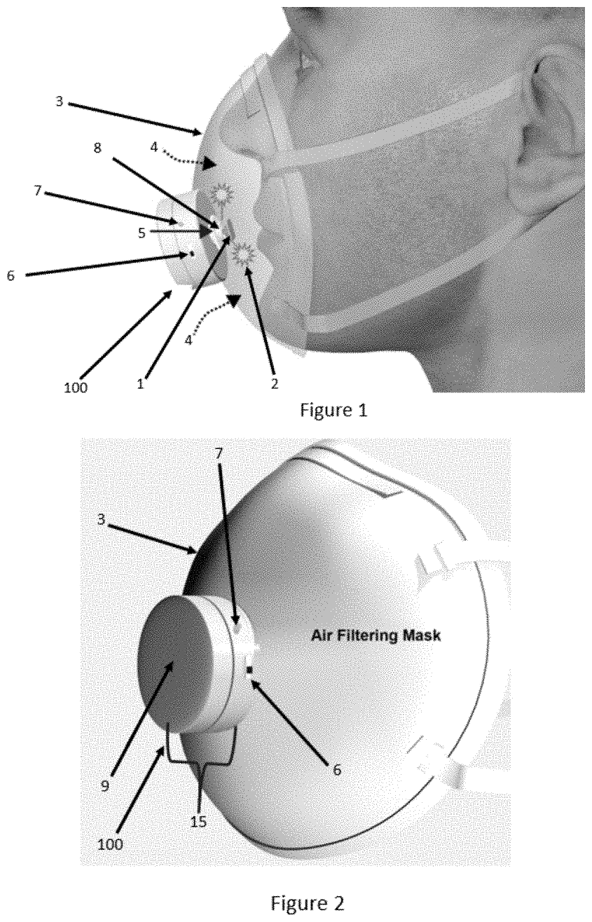

FIG. 1 shows a side view of a face mask 3 with a disinfecting unit 100 according to various exemplary embodiments. The disinfecting unit 100 may be attached in a removeable manner to the face mask 3. The disinfecting unit 100 disinfects air 4 coming in and out of the face mask 3. The disinfecting unit 100 may attach to the face mask 3 with a modular attachment system that utilizes a retainer ring 5 that fastens to a hollow shaft 8 (to be described in greater detail below). In other exemplary embodiments, the disinfecting unit 100 may attach to the face mask 3 using a clipping mechanism. In the exemplary embodiment of FIG. 1, the disinfecting unit 100 is configured to attach to the face mask 3 in the general area of the users mouth when the face mask 3 is being worn. However, it should be understood that the disinfecting unit 100 may be configured to attach to the face mask 3 at any location.

FIG. 1 additionally shows that the disinfecting unit 100 utilizes a reflector 1 to redirect and disperse radiation from the UV LED. The UV LED emits radiation through the hollow shaft 8 until it reaches the reflector 1, which redirects and disperses the radiation to disinfect the air 4 throughout the interior of the face mask 3. The exemplary embodiments of the reflector 1 may be made from a rigid material such as, but not limited to, any plastic, wood, or metal. The rigid material may be then coated with a reflective material such as, but not limited to, silver reflective film, mirror-finish paint, etc. The reflector 1 attaches to the end of the hollow shaft 8 in a removeable manner such as, but not limited to, a screwing or clipping mechanism. For example, the hollow shaft 8 and the reflector 1 may be threaded with mating threads such that the reflector 1 may fasten to the hollow shaft in a similar manner to a nut fastening to a bolt.

FIG. 1 further shows UV radiation killing 2 airborne pathogens, bacteria, and viruses. The exemplary embodiments of the disinfecting unit 100 may be configured to utilize germicidal UV radiation. UV radiation ranging from 260 nm to 270 nm is known as the UVC germicidal bandwidth due to its ability to kill airborne pathogens, bacteria, and viruses. The UV LED used in the exemplary embodiments of the disinfecting unit 100 may radiate in the UVC germicidal bandwidth (e.g., 260-270 nm). Since radiation from the germicidal bandwidth is not visible to the naked eye, the disinfecting unit 100 may include an operating light 7 that notifies the user when the UV LED is operating.

FIG. 2 shows a front view of the disinfecting unit 100, wherein the housing compartment 15 has a cylindrical shape according to various exemplary embodiments. The exemplary embodiments of the housing compartment 15 may have a diameter of 1.00-1.25 inches and a height of 0.50-1.00 inches. However, the housing compartment 15 may be any shape or size so long as the housing compartment 15 is configured to enclose a battery, a circuit, a UV LED, and any other components of the disinfecting unit 100. The components of the disinfecting unit 100 will be described in greater detail below with reference to FIG. 4.

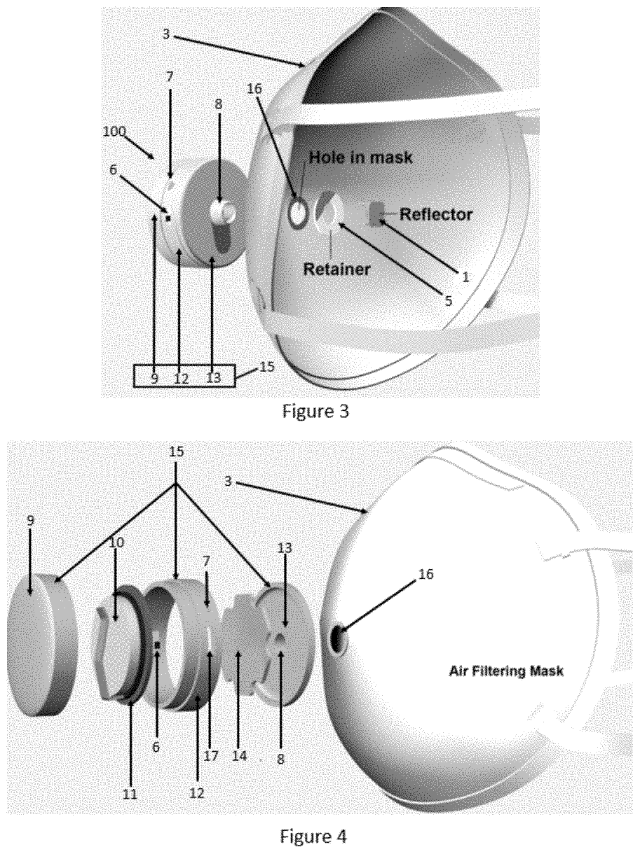

FIG. 3 shows a rear exploded view of a modular attachment system used to removably attach the disinfecting unit 100 to a face mask 3 according to various exemplary embodiments. FIG. 3 shows the hollow shaft 8 may be adapted to be inserted into a hole 16 in the face mask 3 such that the retainer ring 5 and the reflector 1 may be removably attached to the hollow shaft 8 extending from a base cover 13. As shown in FIG. 3, the housing compartment 15 of the disinfecting unit 100 comprises the base cover 13, a center piece 12 and an outer cover 9. The retainer ring 5 may be configured to removably fasten to the hollow shaft 8 with a mechanical mechanism such as, but not limited to, being screwed or clipped on. For example, the hollow shaft 8 may be externally threaded and the retainer ring 5 internally threaded with mating threads, wherein the retainer ring 5 may screw on and off the hollow shaft 8 similar to a nut screwing on and off a bolt. The base cover 13 then connects to the center piece 12, and the center piece 12 connects to the outer cover 9, which allows for the entire disinfecting unit 100 to be removably attached to the face mask 3 through a modular attachment system (e.g., the hollow shaft 8 and the retainer ring 5).

The exemplary embodiments of the hollow shaft 8 may have an outer diameter of 0.125-0.375 inches and a length of 0.25-0.50 inches. In some exemplary embodiments, the face masks 3 may be provided with a hole 16 pre-cut into the face mask 3. In other exemplary embodiments, a user may modify any existing face mask 3 to accept the disinfecting unit 100. For example, prior to attaching the disinfecting unit 100, the user may cut the hole 16 in the face mask 3. The hole 16 may be slightly larger than the outer diameter of the hollow shaft 8 such that the hollow shaft 8 is insertable through the hole 16.

FIG. 4 shows a front exploded view of a disinfecting unit 100 according to various exemplary embodiments. FIG. 4 shows a disinfecting unit 100 adapted to attach to the face mask 3 in a removable manner, wherein a housing compartment 15 of the disinfecting unit 100 may include the outer cover 9, the center piece 12, and the base cover 13 as described above, which may be held together with an adhesive or a mechanical mechanism such as, but not limited to, a screwing or clipping mechanism. FIG. 4 shows the housing compartment 15 may be configured to enclose components of the disinfecting unit 100, which may include, but are not limited to, a battery 10, a circuit 11, and a UV LED 14. The exemplary embodiments of the disinfecting unit 100 may be configured to have the UV LED 14 aligned with open ends of the hollow shaft 8, wherein radiation from the UV LED is transmitted through the hollow shaft 8.

FIG. 4 additionally shows the UV LED 14 is powered by the battery 10 that may be a disposable or rechargeable battery. In the case of a disposable battery, the circuit 11 may be adapted to include a battery port, wherein a disposable battery attaches and detaches as needed. In the case of a rechargeable battery, the circuit 11 may be adapted to include a charging port, wherein a power cord may connect to the circuit 11, through the charge port, to enable charging of the rechargeable battery. The exemplary embodiments of the charge port may be a small port such as, but not limited to, a Micro USB port or USB Type-C port.

The housing compartment 15 is adapted to protect and hold together the battery 10, circuit 11, and UV LED 14. The exemplary embodiments of the housing compartment 15 may be made from any rigid material such as, but not limited to, any plastic, composite or metal. The outer cover 9, center piece 12, and base cover 13 of the housing compartment 15 may be made from the same or different material. The exemplary embodiments may allow for the outer cover 9 to be temporarily removed so the user can remove and replace the battery 10 and other components as needed—such as, but not limited to, the outer cover 9 being unscrewed or unclipped from the center piece 12. Additionally, the exemplary embodiments may have the center piece 12 and the base cover 13 permanently attached or manufactured as one piece.

FIG. 4 further shows the circuit 11 may include a power switch 6. The power switch 6 may attach to the circuit 11 such that it aligns with a slot 17 in the center piece 12. The power switch 6 may be adapted to allow the user to select various operating modes, such as, but not limited to, a continuous power mode or power cycle mode. The continuous power mode would continually power the UV LED 14 while in this mode. Alternatively, the power cycle mode may intermittently power the UV LED 14 in a manner that conserves energy.

FIG. 4 further shows the circuit 11 may include an operating light 7. Because the UV LED radiation is not visible to the naked eye, the operating light 7 may be configured to turn on and off at the same time the UV LED 14 to notify the user when the UV LED 14 is operating. The center piece 12 may be configured with a hole that aligns with the operating light 7, wherein the user can see the operating lighting 7 while using the disinfecting unit 100.

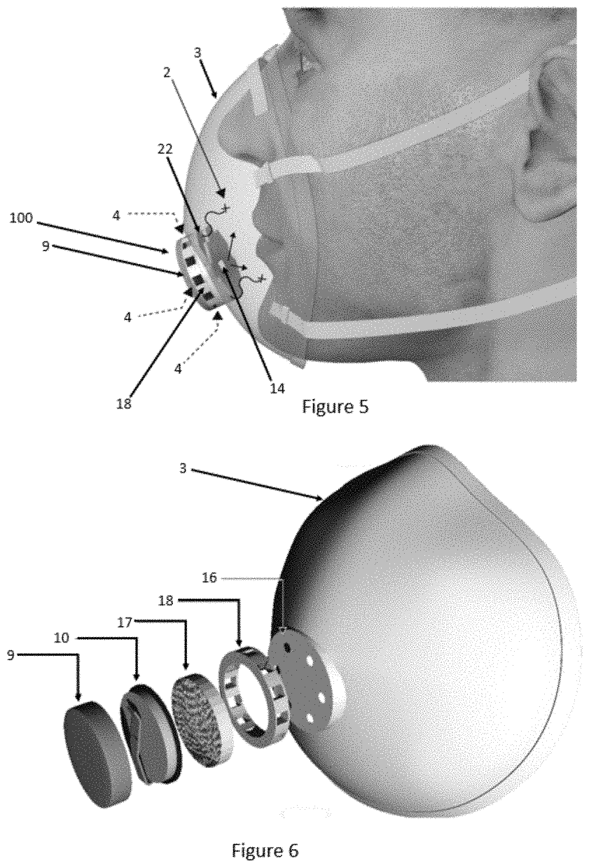

FIG. 5 shows a side view of the disinfecting unit 100 with a filter system attached to a face mask 3 according to various exemplary embodiments. FIG. 5 shows the air 4 flowing into a filter ring 18 and UVC radiation killing 2 any airborne pathogens, bacteria, and viruses that pass through the filter system. As discussed above, the UV LED 14 disinfects airborne contaminants because it radiates in the germicidal bandwidth. The exemplary embodiments of the disinfecting unit 100 with a filter system are configured such that the air 4 flows into the filter system, through the holes 16 in the face mask 3, and then through a gap 22 created by the inner cover. The exemplary embodiments of the inner cover 20 may have a parabolic shape such that a gap 22 is created between the edges of the inner cover 20 and the face mask 3.

FIG. 6 shows a front exploded view of a disinfecting unit 100 adapted to include a filter system according to various exemplary embodiments. The exemplary embodiments of the filter system may include a filter ring 18 and a filter element 17, wherein the filter element 17 is adapted to fit into the center of the filter ring 18. As the user inhales, air 4 flows into the side slots of the filter ring 18, through the filter element 17, and then through the holes 16 and the gap 22, and vice versa for exhaling. The outer cover 9 is adapted to enclose the battery 10 and attach to the top of the filter system in a manner that does not obstruct air 4 flowing through the filter system. The outer cover 9 may be configured to attach to the filter ring 18 by a mechanical mechanism, such as, but not limited to, a screwing or clipping implementation.

The exemplary embodiments of the filter element 17 may include an anti-mildew woven or non-woven material such as, but not limited to, polypropylene, polyester, or polyurethane. Additionally, the exemplary embodiments of the filter element 17 material may be adapted for bi-directional air flow. The filter element 17 is configured to fit into the center of the filter ring 18, wherein the diameter of the filter element 17 is just smaller than the inner diameter of the filter ring 18 and the height of the filter element 17 will be the same height of the filter ring 18. After fitting the filter element into the center of the filter ring 18, the filter element 17 could be secured to the filter ring 18 with any type of adhesive.

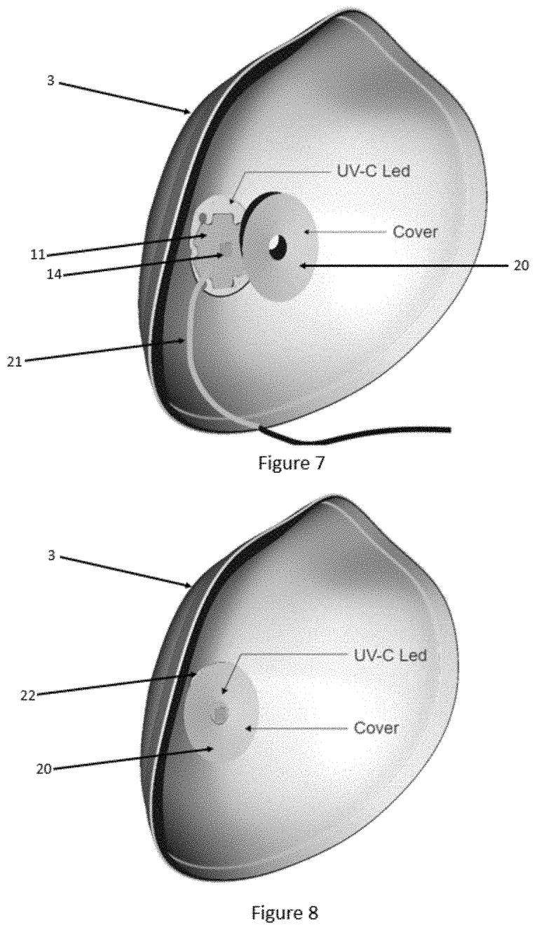

FIG. 7 shows a rear exploded view of a disinfecting unit 100 with a filter system, wherein the circuit 11 is configured to sit under an inner cover 20 according to various exemplary embodiments. As discussed above, the circuit 11 may be configured to operate the UV LED 14 by either constantly powering the UV LED 14 or by cycling the UV LED 14 on and off to conserve energy.

FIG. 7 further shows a power cord 21 configured to connect to the circuit 11 such that it powers the UV LED 14 or recharges the battery 10. The power cord 21 may be temporarily attached (e.g., plugged in) or permanently attached to the circuit 11. The exemplary embodiments of the disinfecting unit 100 could have the power cord 21 permanently attached to the circuit, wherein the power cord 21 would connect to an external power source that would power the UV LED 14. However, the exemplary embodiments of the disinfecting unit 100 may have the power cord 21 configured such that the power cord 21 can plug into a small port incorporated into the circuit 11. The exemplary embodiments of the small port include, but are not limited to, a Micro USB port or USB Type-C port. The circuit 11 may then be configured such that the UV LED 14 is powered by a rechargeable battery, and the power cord 21 is connected when the rechargeable battery needs recharging. The power cord 21 may be configured to be any shape and size. The exemplary embodiments of the power cord 21 may be configured to be a flat cord, wherein the power cord 21 fits nicely between the face mask 3 and the user's face. The power cord 21 may be configured to pass between the face mask 3 and the user's face at any location, for example near the user's chin.

FIG. 8 is a rear view of a face mask 3 that has a disinfecting unit 100 attached to the inside of the mask, wherein the inner cover 20 may be configured to hold the UV LED 14 in place according to various exemplary embodiments. The exemplary embodiments of the inner cover 20 may be configured with a parabolic shape such that there is a gap between itself and the face mask 3, wherein the inner cover does not block the air flowing in and out of the holes 16 in the face mask 3. Because the UV LED 14 sits behind the inner cover 20, the inner cover 20 is adapted with a hole for the UV LED 14 to radiate through. The inner cover 20 could attach to the face mask and the UV LED 14 in a temporary or permanent manner such as, but not limited to, with an adhesive, clip, or Velcro.

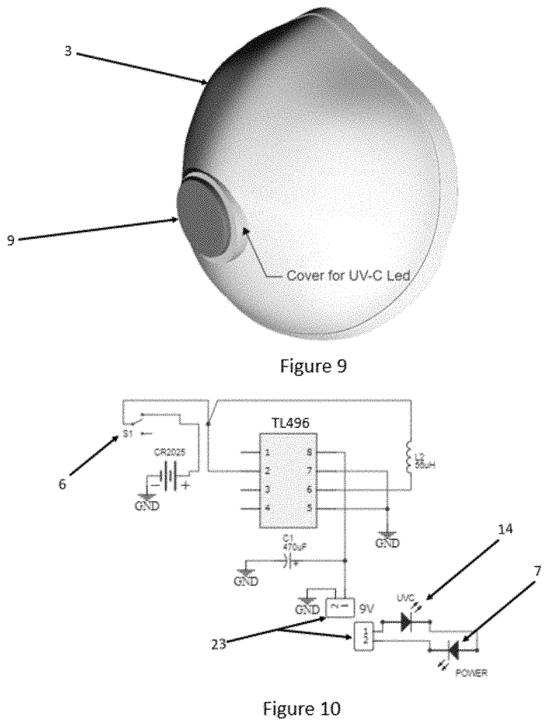

FIG. 9 shows a front view of an outer cover 9 of a disinfecting unit 100 attached to the front of a protective face mask according to various exemplary embodiments. There are no limits or restrictions regarding the shape of the outer cover 9, except the outer cover must be large enough to encase the battery 10 and potentially the circuit 11. FIG. 9 shows the outer cover 9 with a cylindrical shape; however, the shape of the exemplary embodiments of the outer cover 9 may include, but not limited to, a rectangular, hexagonal, or any other shape or design. The shape and size of the outer cover 9 may dictate the shape and size of the rest of the components of the disinfecting unit 100 (e.g., a cylindrical outer cover 9 results in a cylindrical shaped disinfecting unit 100).

FIG. 10 shows a diagram of a circuit that could be used to power a UV LED 14 and an operating light 7 according to various exemplary embodiments. The exemplary embodiments of the circuit may be configured to power the UV LED 14 and the operating light 7 with a disposable battery such as, but not limited to, a CR2025 coin cell battery. The circuit may utilize a TL496 power-supply regulator to increase the CR2025 disposable battery's output from 3 volts to 9 volts, which is needed to power both the UV LED 14 and the operating light 7. The diagram in FIG. 10 shows the UV LED 14 and the operating light 7 could be connected to the circuit through an electrical connector 23. The diagram in FIG. 10 further shows a S1 switch that may be used to turn the circuit on and off, which is a potential configuration of the power switch 6. The exemplary embodiments of the circuit may further include capacitors and inductors such as, but not limited to, a 470 uF capacitor or a 56 uH fixed inductor.

It will be apparent to those skilled in the art that various modifications may be made to present disclosure without departing from the spirit or the scope of the disclosure. Thus, it is intended that the present disclosure cover modifications and variations of this disclosure provided they come within the scope of the appended claims and their equivalent.

What is claimed is:

1. A face mask attachment, comprising:
a disinfecting unit comprising:
an ultraviolet-C light emitting diode, and
a circuit configured to operate the ultraviolet-C light emitting diode, and
a housing adapted to enclose the circuit and the ultraviolet-C light emitting diode; and
a modular attachment system configured to removably attach the disinfecting unit to a face mask.

2. The face mask attachment of claim 1, wherein the disinfecting unit further comprises a battery configured to power the ultraviolet-C light emitting diode, wherein the housing encloses the battery.

3. The face mask attachment of claim 1, wherein the disinfecting unit further comprises:
a filter ring; and
a filter element adapted to fit into the filter ring.

4. The face mask attachment of claim 1, wherein the circuit is configured to connect to a power cord that connects to an external power source to power the ultraviolet-C light emitting diode.

5. The face mask attachment of claim 1, wherein the circuit is configured to cycle the ultraviolet-C light emitting diode on and off to conserve energy.

6. The face mask attachment of claim 1, wherein the modular attachment system comprises:
a hollow shaft that extends from a face center of the housing, wherein the hollow shaft is open on a first end that leads to an interior of the housing and open on a second end that is configured to be inside the face mask when the disinfecting unit is attached to the face mask, wherein the hollow shaft allows the ultraviolet-C light emitting diode in the housing to emit radiation through the hollow shaft into the face mask; and
a retainer ring configured to fasten to the hollow shaft from inside the face mask such that the retainer ring removably attaches the disinfecting unit to the face mask.

7. The face mask attachment of claim 1, wherein the disinfecting unit further comprises a reflector configured to be inside the face mask when the disinfecting unit is attached to the face mask, wherein the reflector reflects the radiation emitted from the ultraviolet-C light emitting diode.

8. A face mask, comprising:
a face covering portion;
a disinfecting unit comprising:
an ultraviolet-C light emitting diode, and
a circuit configured to operate the ultraviolet-C light emitting diode, and
a housing adapted to enclose the circuit and the ultraviolet-C light emitting diode; and
a modular attachment system configured to releasably attach the disinfecting unit to the face covering portion.

9. The face mask of claim 8, wherein the disinfecting unit further comprises a battery configured to power the ultraviolet-C light emitting diode, wherein the housing encloses the battery.

10. The face mask of claim 8, wherein the disinfecting unit further comprises:
a filter ring; and
a filter element adapted to fit into the filter ring.

11. The face mask of claim 8, wherein the circuit is configured to connect to a power cord that connects to an external power source to power the ultraviolet-C light emitting diode.

12. The face mask of claim 8, wherein the circuit is configured to cycle the ultraviolet-C light emitting diode on and off to conserve energy.

13. The face mask of claim 8, wherein the modular attachment system comprises:
a hollow shaft that extends from a face center of the housing, wherein the hollow shaft is open on a first end that leads to an interior of the housing and open on a second end that is configured to be inside the face mask when the disinfecting unit is attached to the face mask, wherein the hollow shaft allows the ultraviolet-C light emitting diode in the housing to emit radiation through the hollow shaft into the face mask; and
a retainer ring configured to fasten to the hollow shaft from inside the face mask such that the retainer ring removably attaches the disinfecting unit to the face mask.

14. The face mask of claim 8, wherein the disinfecting unit further comprises a reflector configured to be inside the face mask when the disinfecting unit is attached to the face mask, wherein the reflector reflects the radiation emitted from the ultraviolet-C light emitting diode.
